(19) United States
(12) Patent Application Publication
(10) Pub. No.: US 2011/0212990 A1
(43) Pub. Date: Sep. 1, 2011

(54) NOVEL POLYMORPH OF MOXIFLOXACIN HYDROCHLORIDE

(75) Inventors: Bandi Parthasaradhi Reddy, Hyderabad (IN); Kura Rathnakar Reddy, Hyderabad (IN); Rapolu Raji Reddy, Hyderabad (IN); Dasari Muralidhara Reddy, Hyderabad (IN); Musku Madhanmohan Reddy, Hyderabad (IN); Deevireddy Bharath Reddy, Hyderabad (IN)

(73) Assignee: HETERO RESEARCH FOUNDATION, Hyderabad, Andhrapradesh (IN)

(21) Appl. No.: 12/678,793

(22) PCT Filed: Nov. 6, 2008
(86) PCT No.: PCT/IN2008/000759

§ 371 Date: Mar. 18, 2010

(51) Int. Cl. A61K 31/437; C07D 471/04; A61P 31/00

(52) U.S. Cl. 514/300; 546/113

(57) ABSTRACT

The present invention relates to novel polymorph of moxifloxacin hydrochloride, processes for its preparation and to pharmaceutical compositions containing it. Thus, for example moxifloxacin hydrochloride is suspended in methanol and water and the p^{H} is adjusted to 1.0-2.0 with concentrated hydrochloric acid at 25° C. and the separated solid is collected and dried to obtain moxifloxacin hydrochloride monohydrate polymorph IV.

### FIELD OF THE INVENTION

The present invention relates to novel polymorph of moxifloxacin hydrochloride, process for its preparation and to pharmaceutical composition containing it.

### BACKGROUND OF THE INVENTION

Moxifloxacin and its salts are antibacterial agents, which were disclosed in EP 550,903. Moxifloxacin, chemically 1-Cyclopropyl-6-fluoro-1,4-dihydro-8-methoxy-7-[(4aS, 7aS)-octahydro-6H-pyrrolo[3,4-b]pyridine-6-yl]-4-oxo-3-quinolinecarboxylic acid, is represented by the following structure:

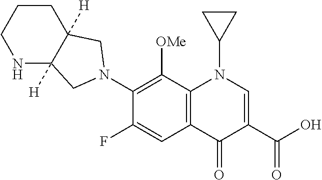

Polymorphism is defined as “the ability of a substance to exist as two or more crystalline phases that have different arrangement and for conformations of the molecules in the crystal Lattice. Thus, in the strict sense, polymorphs are different crystalline forms of the same pure substance in which the molecules have different arrangements and/or different configurations of the molecules”. Different polymorphs may differ in their physical properties such as melting point, solubility, X-ray diffraction patterns, etc. Although those differences disappear once the compound is dissolved, they can appreciably influence pharmaceutically relevant properties of the solid form, such as handling properties, dissolution rate and stability. Such properties can significantly influence the processing, shelf life, and commercial acceptance of a polymorph. It is therefore important to investigate all solid forms of a drug, including all polymorphic forms, and to determine the stability, dissolution and flow properties of each polymorphic form. Polymorphic forms of a compound can be distinguished in the laboratory by analytical methods such as X-ray diffraction (XRD), Differential Scanning Calorimetry (DSC) and Infrared spectrometry (IR).

U.S. Pat. No. 5,849, 752 (“the ‘752 patent”), incorporated by reference, described two crystalline forms of moxifloxacin hydrochloride namely, anhydrous moxifloxacin hydrochloride and monohydrated moxifloxacin hydrochloride. For convenience, the anhydrous crystalline form described in the 752 patent is designated as “Form I”, and the hydrated form as “Form II”. According to U.S. Pat. No. ‘752’, moxifloxacin hydrochloride monohydrate Form II was obtained by stirring a suspension of the anhydrous moxifloxacin hydrochloride in aqueous media until hydration. Moxifloxacin hydrochloride monohydrate of ‘752’ was also prepared by crystallizing moxifloxacin hydrochloride from a media having a water content which is stoichiometrically sufficient but limited to 10%.

WO patent application publication No. 04/091619 disclosed anhydrous Form III of moxifloxacin hydrochloride.

WO patent application publication No. 04/039804 disclosed amorphous form of moxifloxacin hydrochloride.

WO 2005/054240 disclosed two novel crystalline forms which were designated as Form A and Form B of moxifloxacin hydrochloride.

WO patent application publication No. 07/010555 disclosed two crystalline forms which were Form X and Form Y of moxifloxacin hydrochloride. According to WO Publication No. 2007/010555, Form Y was obtained by crystallization of moxifloxacin hydrochloride from the mixture of methanol and water in the ratio of about 8:1 by volume.

WO patent application publication No. 07/148137 disclosed hydrate form of moxifloxacin hydrochloride. According to WO Publication No. 2007/148137, moxifloxacin hydrochloride monohydrate was obtained by crystallization moxifloxacin hydrochloride by humidification of moxifloxacin hydrochloride at 50-90% relative humidity at 25-60° C. for 8 to 24 hours.

WO patent application publication No. 08/028959 disclosed crystalline form of moxifloxacin hydrochloride. According to WO Publication No. 2008/028959, moxifloxacin hydrochloride was obtained by dissolving moxifloxacin hydrochloride in a mixture of methanol and water and adding acetone and recovering moxifloxacin hydrochloride crystalline form.

WO patent application publication No. 08/059521 disclosed process for the preparation of anhydrous crystalline form I of moxifloxacin hydrochloride.

WO patent application publication No. 08/095964 disclosed crystalline form of moxifloxacin base.

We have discovered a stable novel polymorph IV of moxifloxacin hydrochloride monohydrate. The novel polymorph IV is stable over the time and has good flow properties and so, the novel polymorph IV is suitable for formulating moxifloxacin hydrochloride.

One object of the present invention is to provide a stable novel polymorph IV of moxifloxacin hydrochloride monohydrate.

Another object of the present invention is to provide process for preparing the novel polymorph IV of moxifloxacin hydrochloride monohydrate.

Still another object of the present invention is to provide pharmaceutical compositions containing the novel polymorph IV of moxifloxacin hydrochloride monohydrate.

### DETAILED DESCRIPTION OF THE INVENTION

According to one aspect of the present invention there is provided a novel polymorph of moxifloxacin hydrochloride monohydrate designated as polymorph IV is characterized by powder x-ray diffractogram (PXRD) having peaks expressed as 2θ at about 7.5, 9.3, 12.8, 15.1, 16.7, 18.7 and 19.2±0.2 degrees.

PXRD pattern of the moxifloxacin hydrochloride monohydrate polymorph IV of the present invention is shown in FIG. 1.

The water content of the novel polymorph, polymorph IV is in range 3.5 to 4.8 by weight.

The polymorph IV may be identified and differentiated from the known polymorphs by its characteristic PXRD pattern. Thus, for example, a peak at 7.5±0.2 degrees 2θ is present in the PXRD of the polymorph IV of the present invention, but is absent in the PXRD of the moxifloxacin hydrochloride monohydrate disclosed in the U.S. Pat. No. 5,849,752. Similarly, a peak at 18.7±0.2 degrees 2θ is present in the PXRD of the polymorph IV of the present invention, but is absent in the PXRD of the Form Y of moxifloxacin hydrochloride disclosed in the WO Publication No. 2007/010555. Similarly, a peak at 10.3±0.2 degrees 2θ is absent in the PXRD of the polymorph IV of the present invention, but is present in the PXRD of the hydrate Form of moxifloxacin hydrochloride disclosed in the WO Publication No. 2007/148137. Similarly, a peak at 7.5±0.2 degrees 2θ is present and a peak at 8.1±0.2 degrees 2θ is absent in the PXRD of the polymorph IV of the present invention, but the peak at 7.5±0.2 degrees 2θ is absent and a peak at 8.1±0.2 degrees 2θ is present in the PXRD of the moxifloxacin hydrochloride disclosed in the WO Publication No. 2008/028959.

According to another aspect of the present invention there is provided a process for preparation of polymorph IV, which comprises:

a) Preparing moxifloxacin hydrochloride by reacting moxifloxacin free base with hydrochloric acid in a solvent system comprising methanol and water in methanol to water ratio of about 2.3:1 to 4.4:1 by weight; and

b) Isolating the precipitated moxifloxacin hydrochloride monohydrate polymorph IV.

Hydrochloric acid used may be in the form of aqueous hydrochloric acid, hydrogen chloride gas or in the form of hydrochloric acid dissolved in solvent such as methanol.

The weight ratio of methanol to water may preferably be maintained at 2.8:1 to 4:1 and more preferably at 3:1 to 3.8:1.

The temperatures at which moxifloxacin hydrochloride is prepared (step-a) and the moxifloxacin hydrochloride monohydrate is isolated (step-b) are not very critical and the temperature may be maintained in the range 50° C. to −15° C., and also, different temperatures may be maintained during preparation of moxifloxacin hydrochloride and isolation of moxifloxacin hydrochloride monohydrate polymorph IV.

The isolation of moxifloxacin hydrochloride monohydrate polymorph IV may be performed by conventional techniques such as centrifugation and filtration. The preparation of moxifloxacin hydrochloride (step-a) or isolation of moxifloxacin hydrochloride monohydrate polymorph IV may be seeded with moxifloxacin hydrochloride monohydrate polymorph IV.

According to another aspect of the present invention there is provided a pharmaceutical composition comprising moxifloxacin hydrochloride monohydrate polymorph IV.

The pharmaceutical dosage form may preferably be in solid dosage form.

### BRIEF DESCRIPTION OF THE DRAWING

FIG. 1 is a x-ray powder diffraction spectrum of moxifloxacin hydrochloride monohydrate polymorph IV.

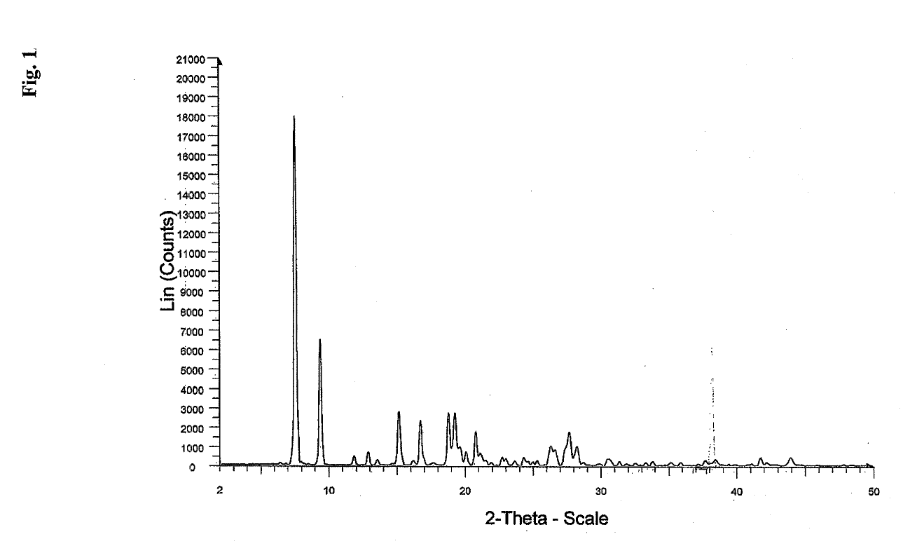

x-Ray powder diffraction spectrum was measured on a Bruker axs D8 advance x-ray powder diffractometer having a copper-Kα radiation.

The invention will now be further described by the following examples, which are illustrative rather than limiting.

### EXAMPLES

### Example 1

a). Preparation of (1-cyclopropyl-6,7-difluoro-8-methoxy-4-oxo-1,4-dihydro-3-quinoline carboxylic acid-O³,O⁴) bis(acyloxy-O) borate.

Acetic anhydride (176.8 gm) is heated to 75° C. and boric acid (30 gm) is added in three lots at 75-90° C. The reaction mass is then stirred at 140° C. for 1 hour and cooled to 70-75° C. Ethyl 1-cyclopropyl-6,7-difluoro-1,4-dihydro-8-methoxy-4-oxoquinoline-3-carboxylate (100 gm) is added and the reaction mass is maintained at 100-105° C. for 1 hour. The reaction mass is then cooled to 0° C., water (1000 ml) is added at 0-5° C. and stirred for 2 hours at 0-5° C. The solid obtained is collected by filtration and the solid is dried at 55-60° C. to obtain 125 gm of (1-cyclopropyl-6,7-difluoro-8-methoxy-4-oxo-1,4-dihydro-3-quinoline carboxylic acid-O³,O⁴) bis(acyloxy-O) borate.

### b). Preparation of Moxifloxacin Hydrochloride Monohydrate Polymorph IV

The mixture of (S,S)2,8-diazabicyclo[4,3,0]nonane (32.8 gm), (1-cyclopropyl-6,7-difluoro-1,4-dihydro-8-methoxy-4-oxoquinoline-3-carboxylic acid-O³,O⁴) bis (acyloxy-O)borate (100 gm), 1,8-diazabicyclo[5,4,0]undec-7-ene (12.6 gm) and toluene (500 ml) is heated to 75 -80° C. for 1 hour 30 minutes. Distilled off the solvent completely under vacuum and methanol (400 ml) and water (100 ml) are added to reaction mass. The p^{H} is adjusted to 1.0-2.0 with concentrated hydrochloric acid at 25° C. and cooled to 5° C. The solid obtained is collected by filtration and the solid is dried at 60-65° C. for 5 hours to obtain 72.5 gm moxifloxacin hydrochloride monohydrate polymorph IV.

The product obtained above may further be processed, if required, to obtain moxifloxacin hydrochloride monohydrate polymorph IV in higher chromatographic purity as follows:

The product obtained above (50 gm) is suspended in water (600 ml). The p^{H} is adjusted to 7.5-8.0 with 50% aqueous sodium hydroxide solution and extracted moxifloxacin free base with methylene dichloride (500 ml). Distilled off the methylene dichloride layer to get moxifloxacin free base as oily residue. To the oily residue, methanol (360 ml) and water (100 ml) are added and the contents are heated to 60° C. The clear solution obtained is subjected to carbon treatment and the reaction mass is filtered through cellite bed and the bed is washed with hot methanol and water mixture (50 ml, 4:1 by volume). The filtrate is cooled to 25° C. The p^{H} is adjusted to 1.0-2.0 with concentrated hydrochloric acid at 25° C. and cooled to 0° C. The solid obtained is collected by filtration and the solid is dried at 60-65° C. for 5 hours to obtain 46 gm of moxifloxacin hydrochloride monohydrate polymorph IV.

### Example 2

Anhydrous moxifloxacin hydrochloride (10 gm) is suspended in water (120 ml). The p^{H} is adjusted to 7.5-8.0 with 50% aqueous sodium hydroxide solution and extracted moxifloxacin free base with methylene dichloride (100 ml). Distilled off the methylene dichloride layer to get moxifloxacin free base as oily residue. To the oily residue, methanol (75 ml) and water (20 ml) are added and the contents are heated to 60° C. The clear solution obtained is subjected to carbon treatment and the reaction mass is filtered through cellite bed and the bed is washed with hot methanol and water mixture (10 ml, 4:1 by volume). The filtrate is cooled to 25° C. The p^{H} is adjusted to 1.0-2.0 with concentrated hydrochloric acid at 25° C. and cooled to 0° C. The solid obtained is collected by filtration and the solid is dried at 60-65° C. for 5 hours to obtain 9.2 gm of moxifloxacin hydrochloride monohydrate polymorph IV.

### Example 3

Moxifloxacin hydrochloride monohydrate (10 gm) as disclosed in the U.S. Pat. No. 5,849,752 is suspended in methanol (70 ml) and water (20 ml). The p^{H} is adjusted to 7.5-8.5 with 50% aqueous sodium hydroxide solution. The clear solution obtained is subjected to carbon treatment and the reaction mass is filtered through cellite bed and the bed is washed with hot methanol and water mixture (10 ml, 4:1 by volume). The filtrate is cooled to 25° C. The p^{H} is adjusted to 1.0-2.0 with concentrated hydrochloric acid at 25° C. and cooled to 0° C. The solid obtained is collected by filtration and the solid is dried at 60-65° C. for 6 hours to obtain 9.4 gm of moxifloxacin hydrochloride monohydrate polymorph IV.

We claim:

1. A polymorph IV of moxifloxacin hydrochloride monohydrate, characterized by an X-ray powder diffractogram having peaks expressed as 2θ angle positions at about 7.5, 9.3, 12.8, 15.1, 16.7, 18.7 and 19.2±0.2 degrees.

2. A process for preparation of polymorph IV of moxifloxacin hydrochloride monohydrate as defined in claim 1, which comprises:
a. Preparing moxifloxacin hydrochloride by reacting moxifloxacin free base with hydrochloric acid in a solvent system comprising methanol and water in methanol to water ratio of about 2.3:1 to 4.4:1 by weight; and
b. Isolating the precipitated moxifloxacin hydrochloride monohydrate polymorph IV.

3. The process as claimed in claim 2, wherein the hydrochloric acid used may be in the form of aqueous hydrochloric acid, hydrogen chloride gas or in the form of hydrochloric acid dissolved in solvent such as methanol.

4. The process as claimed in claim 2, wherein the weight ratio of methanol and water preferably maintained at 2.8:1 to 4:1.

5. The process as claimed in claim 4, wherein the weight ratio of methanol and water more preferably maintained at 3:1 to 3.8:1.

6. The process as claimed in claim 2, wherein the temperature at which moxifloxacin hydrochloride is prepared (step-a) is in the range of 50° C. to −15° C.

7. The process as claimed in claim 2, wherein the temperature at which moxifloxacin hydrochloride monohydrate is isolated is in the range of 50° C. to −15° C.

8. The process as claimed in claim 2, wherein (step-a) or (step-b) is seeded with moxifloxacin hydrochloride monohydrate polymorph IV.

9. A pharmaceutical composition comprising moxifloxacin hydrochloride monohydrate polymorph IV of claim 1 and a pharmaceutically acceptable excipient.

10. The pharmaceutical composition as claimed in claim 9, wherein the pharmaceutical composition of moxifloxacin hydrochloride monohydrate polymorph IV is a solid dosage form.
